(19) United States
(12) Patent Application Publication
(10) Pub. No.: US 2008/0057007 A1
(43) Pub. Date: Mar. 6, 2008

(54) ORAL HYGIENE PRODUCTS CONTAINING ASCORBIC ACID AND METHOD OF USING THE SAME

(75) Inventors: Charles LEONHARDT, Huntington, NY (US); John C. BUSBY, Penndel, PA (US)

Correspondence Address: CAESAR, RIVISE, BERNSTEIN,;COHEN & POKOTILOW, LTD., 11TH FLOOR, SEVEN PENN CENTER, 1635 MARKET STREET, PHILADELPHIA, PA, 19103-2212, US

(73) Assignee: DENTECH, INC., Penndel, PA (US)

(21) Appl. No.: 11/874,637

(22) Filed: Oct. 18, 2007

Related U.S. Application Data
(63) Continuation-in-part of application No. 11/365,167, filed on Mar. 1, 2006.

(51) Int. Cl. A61K 8/21; A61C 15/00; A61K 8/365; A61K 8/46; A61P 43/00

(52) U.S. Cl. 424/52; 424/55; 424/57; 433/216

(57) ABSTRACT

The present invention is directed to dental compositions, including dentifrices, containing ascorbic acid for removing and inhibiting dental biofilms which form plaque and tartar, and also for treating and preventing gingivitis and periodontitis. The ascorbic acid composition can contain may additional ingredients, including an enamel-strengthening component, and be used in many different forms, including breath spray, chewing gum, dental floss, dental powder, gargle, lozenge, mouth spray, mouth wash, tooth gel, tooth liquid, tooth paste and tooth strips. Also described in a method of using a dental composition containing ascorbic acid in order to treat plaque and tartar as well as gum disorders.

### BACKGROUND OF THE INVENTION

The present invention relates generally to compositions and methods useful in promoting oral health and hygiene and, in particular, to dental compositions comprising ascorbic acid for removing and inhibiting dental biofilm, plaque and tartar.

Bacteria are the primary etiologic agents in periodontal disease. Tooth decay and gum inflammation are often the result of microbial plaque activity, which includes bacterial products, leukocytes, epithelial cells and saliva components. In fact, more than 500 bacterial strains may be found in dental plaque. In the presence of saliva, proliferating bacteria attach to places with retained food such as the gum line, tongue, tooth spacing, pits and fissures. These bacteria have also evolved to survive in the environment of the tooth surface, gingival epithelium, and oral cavity. These bacteria decompose retained food, releasing toxic substances and forming plaque and tartar (an advanced form of plaque), together often referred to as dental calculus. This results in bad breath, tooth decay, gum inflammation and dental caries. Recent advances have led to the recognition that dental plaque is a biofilm and changes in thinking about the structure of dental plaque has led to an improved understanding of why periodontitis is so difficult to treat.

The majority of oral care products contain a large percentage of artificial ingredients which can be harmful when ingested. Some of the products induce allergic reactions and others are even carcinogenic when used in high dosages. Antibiotics have also been used to fight plaque formation. However, antibiotic applications usually result in the development of resistant microorganisms. As a consequence, there is a trend toward the use of safer ingredients in oral care products.

Oral care products containing safe ingredients, such as baking soda, are well known. However, high concentration of baking soda required to provide adequate cleaning is abrasive and distasteful. Saponin is another type of safe plaque cleanser, which produces foaming and cleans without the use of artificial surfactants. However, saponins are of plant origin (Quillaja and/or Yucca tree) and have to be extracted from plants, which is a laborious and time consuming process.

Ascorbic acid (vitamin C) is a safe ingredient and has been used in oral compositions. However, those compositions usually contain a small amount of ascorbic acid and mainly rely on other cleaning ingredients. For example, Japanese Patent 2005320321 A describes the use of a dentifrice composition comprising 0.01% to 15% ascorbic acid and hydroxyapatite; however, due to its low concentration of ascorbic acid, this composition only whitens teeth and does not treat and remove plaque and tartar from surfaces in the mouth. Other compositions use ascorbic acid but fail to efficiently utilize its strong and safe cleaning capacity heavily relying on catalysts for auto-oxidation, such as copper, and the synergetic action of other cleaning agents. None of the above prior art describes, suggests or renders obvious the enormous cleaning capacity of ascorbic acid crystals and/or granules unaided by other cleaning agents.

For the past several decades, the emphasis in oral hygiene has been placed on attempts to develop products and methods for removing plaque and tartar. To date, these attempts, and advances thereto, have experienced moderate success. There is, however, still a need for improved oral hygiene products and methods for inhibiting and removing dental calculus, especially tartar, and for preventing mouth disorders and disease.

More recently, advances in research technology have allowed researchers to study bacteria in their natural environment. These studies revealed that most bacteria live in complex communities called biofilms which adhere to surfaces in the mouth and are embedded in an extracellular slime layer. Once the bacterium attaches to a surface, it activates a whole different set of genes that gives the bacterium different characteristics from those that it had as a free-floating organism. It is now known that a biofilm community comprises bacterial microcolonies, an extracellular slime layer, fluid channels, and a primitive communication system. Dental bacterial plaque is a biofilm that adheres tenaciously to tooth surfaces, restorations, and prosthetic appliances as well as other surfaces in the mouth and throat regions of the body. Understanding the formation, composition, and characteristics of dental biofilm assists in its removal and control. Therefore, a need exists for an improved dental composition for removing dental biofilm in order to prevent the accumulation of plaque and tartar in the mouth.

The present invention relates to a safe dental composition or dentifrice for treating dental biofilm, plaque and tartar. The invention also relates to the use of the dental composition for removing and inhibiting the formation of dental biofilm, plaque and tartar in a subject's mouth. The invention is highly efficient in plaque and tartar removal and inhibition and is also a natural tooth whitener and preservative.

### BRIEF SUMMARY OF THE INVENTION

The invention is a dental composition comprising ascorbic acid as well as a method for using the composition in order to remove and inhibit dental biofilm, plaque and/or tartar in the mouth and throat regions of a body. There are several variations of the dental composition and the method of using same.

While the prior art avoids using high concentrations of ascorbic acid as harmful to tooth enamel, the present invention is a dental composition which contains a high weight concentration of ascorbic acid in order to effectively remove dental calculus from the mouth and throat regions of a body. The dental composition can contain additional ingredients including an enamel-strengthening component (e.g., hydroxyapatite) which will effectively and efficiently protect tooth enamel. If desired, additional ingredients can also be added to the composition of the invention including sweeteners, flavoring and coloring agents to name only a few. Also, the composition of the invention can be used in a variety of commercial products such as toothpaste, chewing gum, mouthwash and mouth spray, to name only a few, in order to cover a wide range of consumer needs.

The method of the present invention is directed to the use of a dental composition comprising ascorbic acid such that the composition is taken into the mouth for a sufficient period of time to begin to attack and/or remove dental biofilm, plaque and/or tartar and strengthen enamel on teeth after which the composition is expectorated from the mouth. The method also has the added advantage of removing dental biofilm from other surfaces of the mouth and throat regions, including the larynx and vocal cords.

It is an object of the present invention to provide an improved dental composition for removing biofilm, plaque and/or tartar from a user's mouth and throat regions of the body. It is also an object of the present invention to provide a method for removing, controlling and/or inhibiting biofilm, plaque and/or tartar in a user's mouth. It is another object of the present invention to provide a safe oral hygiene composition which promotes human and animal health. To this end, it is yet another object of the present invention to prevent mouth and gum disorders such as caries, gingivitis, and other periodontal diseases. Still another object of the present invention is to provide a safe, healthy and efficient cleaning procedure for especially children by eliminating the need for long and extensive brushing. Yet another object of the invention is to provide an improved dental composition and method of using same which is convenient, portable and ingestible.

### DETAILED DESCRIPTION OF THE INVENTION

The invention is based on the enormous cleaning capacity of ascorbic acid crystals, granules and/or any other form of ascorbic acid unaided by any other cleaning ingredients. Preferably, ascorbic acid is in a crystalline form. The use of the dental composition is not limited to humans but can be effective in many other subjects, including animals that experience the formation of dental biofilm, plaque and/or tartar in their mouth and throat areas. More specifically, the present invention is directed to a dental composition for removing and inhibiting at least one of dental biofilm, plaque and tartar comprising an effective amount of ascorbic acid. The composition is also effective in treating and/or preventing teeth and gum disorders.

Ascorbic acid is a non-toxic compound which is harmless when ingested. The molecular structure of ascorbic acid is Vitamin C which is required for the growth and repair of tissues in all parts of your body. It is necessary to form collagen, an important protein used to make skin, scar tissue, tendons, ligaments, and blood vessels. Vitamin C is also essential for the healing of wounds, and for the repair and maintenance of cartilage, bones, and teeth.

Vitamin C deficiency can lead to dry and splitting hair; gingivitis (inflammation of the gums) and bleeding gums as well as rough, dry, scaly skin; decreased wound-healing rate, easy bruising; nosebleeds; weakened enamel of the teeth; swollen and painful joints; anemia; decreased ability to ward off infection; and, possibly, weight gain because of slowed metabolic rate and energy expenditure. A severe form of vitamin C deficiency is known as scurvy, which mainly affects older, malnourished adults. The body does not manufacture vitamin C on its own, nor does it store it. It is therefore important to include plenty of vitamin C-containing foods in a daily diet. Thus, ascorbic acid is not only harmless but also important for healthy functioning. Most preferably, the present invention uses high concentrations of ascorbic acid in combination with an enamel-strengthening component to clean teeth and protect tooth enamel.

Dental plaque is a biofilm which simply stated is a fatty substance consisting of bacterial colonies surrounded by a gel-like intercellular substance derived chiefly from the bacteria themselves. Plaque also contains saliva, epithelial cells and leukocytes. It usually accumulates on the surface of teeth, gums, gum lines, on the tongue and in the throat region resulting in bad breath, tooth decay, gum disorders and caries. Bacterial colonies of the plaque use dietary carbohydrates as a source of energy producing acids. The acids demineralize tooth enamel and dentine attacking gum tissue and reacting with the calcium in the teeth. Different studies have confirmed the role of microbial plaque as a major factor in dental caries and periodontal diseases.

The most common types of periodontal disease are gingivitis and periodontitis. Gingivitis is an early stage gum disease characterized by gum inflammation, swelling and bleeding. Periodontitis is a late stage gum disease, in which tooth supporting bone is slowly lost and, if left untreated, can result in tooth loss. In view of dental biofilm and plaque's major role in dental diseases, one of the objects of the present invention is to provide safe and effective oral hygiene composition for combating bacteria associated with dental plaque, caries, and periodontal diseases. In one embodiment of the present invention, the dental composition of the invention breaks down the dental biofilm barrier formed, for example, on the surface of a tooth so that antibacterial agents in the composition can attack bacterial colonies that create plaque and, eventually, tartar. Accordingly, the present invention provides an improved dental composition for relatively fast and efficient removal and inhibition of dental biofilm and plaque thereby preventing and treating periodontal disease, including gingivitis and periodontitis.

The present invention also efficiently attacks, removes and inhibits tartar. Tarter is generally considered to be an advanced form of plaque which forms by a complex biological process. Very simply, tartar is formed when inorganic salts and phosphates in saliva deposit on plaque, calcify and form a hard, strong surface. Relative to plaque, tarter is difficult to remove once formed and thus is usually removed by mechanical means such as ultrasonic scrapers, picks and brushes. Tartar that remains on teeth for a long time period of time may result in serious tooth and gum disorders. While the dental composition of the present invention has some abrasive features, the ascorbic acid effectively breaks down tarter by primarily a chemical means. As a result, each application of the dental composition of the invention reduces and inhibits tartar formation thereby resulting in the prevention of dental caries and periodontal diseases.

The dental composition of the present invention and, in particular, a relatively high concentration of ascorbic acid, attacks dental biofilm which leads to the formation of dental calculus in the mouth and throat areas of a subject. It is believed that dental biofilm differs from subject to subject depending on body chemistry including, among other things, the pH of saliva. Typically, the solubility of plaque is about pH 5.5. As a result, it is preferred that an aqueous solution containing the dental composition of the present invention has a pH of about 4.5 to about 5.5. This can be accomplished, for example, by combining a sufficient quantity of the dental composition with saliva in a mouth so that a pH of less than about 5.5 is achieved in the mouth and throat regions as this will begin to solubilize plaque in these regions of the body. Thus, preferably, the pH of the dental composition of the present invention is less than about 5.5.

Ascorbic acid crystals and/or granules in the form of dental powder can be viewed as the most efficient embodiment of the invention. Preferably, the dental composition of the present invention comprises from about 15% to about 100% ascorbic acid, most preferably the dental composition comprises greater than 50% ascorbic acid by weight. The present invention can also be used in other forms, including a liquid form as an aqueous and/or alcohol solution. However, forms other than dental powder are expected to contain a lower concentration of ascorbic acid as the concentration will be limited by the amount of ascorbic acid that will enter a solution. For example, it is expected that an aqueous solution of the dental composition will contain a maximum of about 45% ascorbic acid.

Also, in addition to a dental powder, the dental composition of the present invention can be a breath spray, chewing gum, gargle, lozenge, mouth spray, mouth wash, tooth gel, tooth liquid, and toothpaste. The toothpaste form can be a water-free paste such as a water-free glycerol paste and aqueous forms of the dental composition can be made with or without alcohol. The aqueous solutions can contain up to about 80% water and the alcohol solutions can contain up to about 30% denatured alcohol and up to about 50% water. The alcohol used can be, but is not limited to, thymol and menthol.

In other embodiments of the present invention the dental composition is impregnated in a dental tool. Such dental tools include, but are not limited to, a toothbrush, tooth strips, dental floss, and dental instruments. These tools function such that, when used in a mouth, the dental composition contacts saliva, goes into solution, and begins to attack dental biofilm, plaque and tartar.

The toothpaste, dental powder and mouthwash forms of the invention are more suitable for use in domestic or household settings, where they are applied to teeth with or without a brush. For example, after toothpaste application and brushing of a subject's teeth and mouth regions are completed, the mouth can be easily rinsed with water and the water and toothpaste expectorated from the mouth. Similarly, the mouthwash form of the present invention can be comfortably used in a domestic or household setting where a person has an opportunity to extensively rinse his or her oral cavity. After the rinse is completed, the mouthwash form of the present invention is expectorated and, if desired, the oral cavity is further rinsed with, for example, water. Importantly, any accidental swallowing will not be harmful considering that the dental composition is ingestible.

In contrast to the toothpaste, dental powder and mouthwash, which are more suitable for domestic use, the mouth spray and chewing gum forms of the invention can be universally used. Although many people are willing to take oral hygiene measures throughout the day, they often find those measures to be inconvenient, and sometimes awkward. For example, they may not have constant access to a bathroom or a sink and, therefore, are unable to use toothpaste or mouthwash. Also, they may be uncomfortable being seen carrying around items such as a toothbrush, toothpaste or a bottle of mouthwash. Consequently, there is a great need for oral hygiene products, like the mouth spray and gum forms of the present invention, which are convenient, portable and ingestible.

The mouth spray of the present invention merely requires spraying a solution of the dental composition into a person's mouth and retaining it in the mouth for an appropriate amount of time such that the composition begins to remove dental biofilm, plaque and/or tartar. Typically, the dental composition needs to remain in the mouth for less than about 2 minutes in order to ensure its effectiveness. After that time period has passed, the dental composition can be safely swallowed or simply expectorated from the mouth. It is envisioned that mouth sprays of the present invention will be packaged in portable bottles in order to fit into pockets, purses and bags.

The chewing gum form of the present invention is another effective form of the present invention. Plaque and even tarter can be dislodged or otherwise removed from mouth surfaces by chewing the gum for sufficient period of time. Furthermore, a person can continue chewing the gum even after plaque is dislodged or removed in order to inhibit plaque accumulation and tarter formation over longer time periods.

Preferably, the size of the ascorbic acid particles is greater than about 5 microns. Most preferably, the particle size is about 5 microns to about 100 microns. When used in high concentrations (ranging from about 15% to about 100% by weight), the ascorbic acid particles have enormous cleaning capacity eliminating the need for other cleaning agents and, very possibly, further mechanical cleaning. Such high concentrations are extremely effective in killing a wide spectrum of bacteria such as oral microflora, including: Actinomyces viscosus, alpha Streptococcus, Candida albicans, Escherichia coli, Pseudomonas aeruginosa, Staphylococcus epidermidis and Streptococcus mutants. Notwithstanding, if desired, the dental composition of the present invention may contain at least one antibacterial ingredient for exterminating bacteria that lead to the formation of plaque and tartar.

Once in a mouth, the dental composition of the present invention combines with saliva to form a solution and begins to remove dental biofilm from teeth, gums and gum lines, as well as the tongue and throat regions by both chemical and mechanical action. Similarly, liquid, gel, paste, spray and other forms of the dental composition are capable of removing dental biofilm from these various mouth and throat regions of the body. The amount of time that the dental composition must remain in the mouth to be effective will depend on the concentration of the composition and the form of the product which contains the composition. For example, many dental powder forms of the invention will usually begin to remove plaque and tartar in less than about 60 seconds; whereas a similar concentration of the composition in a toothpaste form can take slightly longer.

The dental composition of the present invention can also remove dental biofilm and calculus from between teeth, and in other small crevices in the mouth, depending on the size of the opening or crevice and the strength of the composition. The invention often eliminates the need for additional cleaning agents and the mechanical action of scrapers and other dental tools on exposed surfaces in the mouth and throat regions. However, if necessary or desired, dental tools can be used in combination with the claimed invention in order to remove calculus from areas in the mouth which are not readily exposed. Further, the amount and/or size of the ascorbic acid particles can be varied in order to comfortably achieve the desired taste and effectiveness. Moreover, the invention is highly efficient in preventing plaque accumulation and tartar formation if used consistently.

Individuals usually brush their teeth for less than 60 seconds during each brushing session thereby limiting the exposure time teeth have to the chemical and mechanical actions caused by toothpaste and a toothbrush. Most dentists and oral hygienists recommend longer treatments for efficient plaque removal. The dental composition of the present invention works quickly to remove dental biofilm, plaque and/or tartar, resulting in smooth teeth surfaces as well as clean gums, tongue and throat regions. Furthermore, ascorbic acid softens plaque formed between teeth and loose gums. Plaque accumulation is also inhibited in these areas resulting in tighter gum lines and fresher breath. As a consequence, dental caries and periodontal diseases are effectively prevented.

By removing plaque and tartar from teeth in a mouth, the dental composition may eventually expose some enamel on the teeth. Because there is some evidence that long exposure of high concentrations of ascorbic acid may be harmful to tooth enamel, the dental composition of the present invention can also comprise an enamel-strengthening component to protect the enamel. The enamel-strengthening component can be any component known or yet to be discovered that protects tooth enamel. Preferably, the enamel-strengthening component is hydroxyapatite, a phosphate compound, or a fluoride compound. For example, the phosphate compound can be sodium monofluorophosphate and the fluoride compound can be ammonium fluoride, sodium fluoride or stannous fluoride. It is also preferred that the concentration of the enamel-strengthening component is less than about 1.0% by weight, most preferably, less than about 0.5% by weight. The enamel-strengthening component adheres to exposed surfaces in the mouth and promotes the recalcification and strengthening of teeth. The enamel-strengthening component is capable of being used effectively in dental filling methods for protecting, restoring and/or repairing pits, fissures and lesions in tooth enamel. Preferably, the enamel-strengthening components of the present invention also effectively absorb dental plaque.

As a result of containing an effective amount of an enamel-containing component, the composition of the present invention will not compromise, and instead will tend to repair, tooth enamel. Additionally, in one exemplary embodiment of the present invention, any mouth irritation, such as oral mucosa, that may be caused by the ascorbic acid can be easily treated by adding menthol to the dental composition.

In addition to containing an effective amount of ascorbic acid and an enamel-containing component, the dental composition of the present invention can, if desired, contain other ingredients including one or more of an abrasive agent, antibacterial agent, alcohol, bioactive material, carrier material, cellulose, coloring agent, filler material, fluoride, flavoring agent, glycerin, menthol, phosphate, silica, sodium benzoate, sodium carbonate peroxide, sodium saccharine, sweeteners, triclosan, thymol, water, whitening agent and zinc citrate.

For example, the dental composition can contain sodium bicarbonate and/or pumice where additional abrasiveness is desired. If desired, sweeteners, flavoring and coloring agents can also be added to achieve different tastes and flavors. Suitable sweeteners include, but are not limited to, sodium saccharine, aspartame, cyclamates, sucrose, sorbitol, mannitol, and maltitol. The preferred sweetener is sodium saccharine (0.01-0.02%). Also, suitable flavoring agents include both natural and synthetic oils such as cinnamon oil, wintergreen oil, bay oil, citrus oil, lemon oil, lime oil and clove oil. Preferably, the dental composition is flavored with spearmint or peppermint in an amount of about 0.2%-0.4% by weight and, most preferably, spearmint and peppermint are combined in an amount of about 0.2%-0.4% by weight.

Bioactive ingredients or medications include, but are not limited to, antifungal, anti-inflammatory, antibiotic, anti-bacterial, analgesic and immunosuppressive agents. Also, ascorbic acid is a natural whitener, eliminating the need for additional whitening agents. However, if desired, the dental composition of the present invention can include such additional whitening ingredients as sodium carbonate peroxidase (about 3-5%) and hydrated silica (about 5-7%). Further, it is preferred that, when triclosan is added to the dental composition, its concentration be about 0.1% to about 0.5% by weight, most preferably 0.3% by weight.

The harmless nature of the present invention makes it even more suitable for use by children. Since many children do not regularly or efficiently brush their teeth, the present invention solves this problem by providing a safe, healthy and efficient dental composition and cleaning procedure eliminating the need for long and extensive brushing.

Since ascorbic acid is a preservative, the dental composition of the present invention need not be specially stored or packaged in sealed containers. Given the invention's preserving characteristics, its solutions can be made with tap water without compromising the solutions' cleaning properties; however, purified water is preferred. Also, the dental composition of the invention can contain additional preservatives such as sodium benzoate in an amount of about 0.2-0.4%.

In addition to dental compositions, the present invention is also directed to a dental method comprising the steps of taking into a mouth an effective amount of a dental composition comprising ascorbic acid; maintaining the dental composition in the mouth for a sufficient period of time; and expectorating the dental composition from the mouth. If desired, the dental method can further include rinsing the mouth with, for example, water or another form of mouth wash; and expectorating the rinse from the mouth. Additionally, various dental tools, including those previously described, can be employed with this dental method. These steps can also be repeated and, if performed on a consistent basis, will effectively remove and inhibit the formation of dental calculus in a mouth and throat region of a body. This same method can also be used effectively to treat gum disorders, prevent gum disorders, or both. Such gum disorders include, but are not necessarily limited to, caries, gingivitis and periodontitis.

### EXAMPLES

The invention is now described in further detail with respect to the following examples. The examples are only illustrative examples, containing approximate percentages of various ingredients, and are not intended to be considered as limitations of the invention.

### Example 1

A dental powder containing:

| | |
|---|---|
| 75% | ascorbic acid; |
| 20% | tricalcium phosphate; |
| 0.1-0.2% | menthol; |
| 5% | zinc citrate. |

### Example 2

A medicated chewing gum containing:

| | |
|---|---|
| 69.0% | macticatory gum core; |
| 20.0% | ascorbic acid; |
| 0.1% | sodium saccharine; |
| 0.5% | hydroxyapatite; |
| 0.2% | spearmint/peppermint flavor; |
| 0.24% | sodium fluoride; |
| 0.001% | blue #1; |
| 5.0% | zinc citrate trihydrate; |
| 2.0% | sucralose; |
| 1.0% | polymer coating for sugar-free chiclets; |
| 0.1% | sodium benzoate; |
| 1.0% | titanium dioxide; |
| 0.85% | other ingredients. |

### Example 3

The tooth powder containing:

| | |
|---|---|
| 70.0% | ascorbic acid; |
| 12.0% | tricalciumphosphate or dicalciumphosphate; |
| 0.1% | menthol natural crystalline powder; |
| 0.2% | spearmint/peppermint flavor; |
| 0.24% | sodium fluoride; |
| 3.0% | sodium carbonate peroxide; |
| 5.0% | hydrated silica; |
| 5.0% | zinc citrate trihydrate; |
| 2.0% | sucrose; |
| 0.5% | hydroxyapatite; |
| 1.0% | titanium dioxide; |
| 0.96% | other ingredients. |

### Example 4

An alcohol-free mouthwash containing:

| | |
|---|---|
| 25.0% | ascorbic acid; |
| 0.01% | sodium saccharine; |
| 0.2% | spearmint/peppermint flavor; |
| 0.001% | blue #1; |
| 5.0% | zinc citrate; |
| 0.7% | cethylpyridium chloride; |
| 10.0% | glycerin; |
| 0.1% | polymer 407; |
| 0.5% | hydroxyapatite; |
| 58.0% | deionized water; |
| 0.48% | other ingredients. |

### Example 5

A mouthwash containing:

| | |
|---|---|
| 20.0% | ascorbic acid; |
| 10.0% | sorbital solution; |
| 0.01% | sodium saccharine; |
| 0.2% | sodium benzoate; |
| 0.2% | spearmint/peppermint flavor; |
| 0.0001% | blue #1; |
| 5.0% | zinc citrate; |
| 0.5% | hydroxyapatite; |
| 10.0% | glycerin; |
| 0.1% | paloxamer 407; |
| 0.24% | sodium fluoride; |
| 0.1% | menthol in denatured alcohol; |
| 0.1% | thymol in denatured alcohol; |
| 20.0% | denatured alcohol with menthol and thymol; |
| 33.0% | deionized water; |
| 0.55% | other ingredients. |

### Example 6

A toothpaste containing:

| | |
|---|---|
| 34.0% | ascorbic acid; |
| 10.0% | sorbital powder; |
| 0.5% | hydroxyapatite crystals; |
| 0.01% | sodium saccharine; |
| 0.2% | sodium benzoate; |
| 0.2% | spearmint/peppermint flavor; |
| 5.0% | zinc citrate trihydrate; |
| 30.0% | glycerin anhydrous; |
| 0.24% | sodium fluoride; |
| 3.0% | calcium orthophates; |
| 5.0% | hydrated silica; |
| 10.0% | tricalcium phosphate; |
| 1.0% | sodium lauryl sulfate; |
| 0.85% | other ingredients. |

Example 7

An antiseptic gargle containing:

| | |
|---|---|
| 28.0% | ascorbic acid; |
| 0.2% | sodium benzoate |
| 0.6% | menthol in denatured |
| 0.05% | methyl salicylate |
| 0.1% | thymol in denatured alcohol |
| 20% | denatured alcohol with menthol and thymol |
| 0.2% | sodium benzoate |
| 1.0% | hydroxyapatite |
| 49.85% | deionized water |

Although the embodiments of the present disclosure have been described with specific examples, it is to be understood that the disclosure is not limited to those specific examples and that various other changes, combinations and modifications will be apparent to one of ordinary skill in the art without departing from the scope and spirit of the invention which is to be determined with reference to the following claims.

We claim:

1. A dental composition comprising an effective amount of ascorbic acid.

2. The dental composition of claim 1, wherein the composition further comprises an enamel-strengthening component.

3. The dental composition of claim 1, wherein the concentration of the ascorbic acid is greater than about 50% by weight of the composition.

4. The dental composition of claim 1, wherein particles of the ascorbic acid are greater than about 5 microns in size.

5. The dental composition of claim 1, wherein the composition has a pH less than about 5.5.

6. The dental composition of claim 2, wherein the enamel-strengthening component is selected from the group consisting of at least one of hydroxyapatite, a phosphate compound, and a fluoride compound.

7. The dental composition of claim 6, wherein the fluoride compound is selected from the group consisting of ammonium fluoride, sodium fluoride and stannous fluoride.

8. The dental composition of claim 6, wherein the phosphate compound is sodium monofluorophosphate.

9. The dental composition of claim 2, wherein the enamel-strengthening component repairs damage to teeth.

10. The dental composition of claim 1, further comprising at least one of the ingredients selected from a group consisting of an abrasive agent, antibacterial agent, alcohol, bioactive material, carrier material, cellulose, coloring agent, filler material, fluoride, flavoring agent, glycerin, phosphate, silica, sodium benzoate, sodium carbonate peroxide, sodium saccharine, sweetener, triclosan, water, whitening agent and zinc citrate.

11. The dental composition of claim 10, wherein the concentration of triclosan is from about 0.1% to about 0.5% by weight of the composition.

12. The dental composition of claim 1, wherein the composition is a component of a dental product selected from the group consisting of breath spray, chewing gum, dental powder, gargle, lozenge, mouth spray, mouth wash, tooth gel, tooth liquid, and toothpaste.

13. The dental composition of claim 1, wherein the composition is impregnated in a dental tool.

14. The dental composition of claim 13, wherein the dental tool is selected from the group consisting of dental floss, a toothbrush, tooth strips and dental instruments.

15. The dental composition of claim 1, wherein the composition treats teeth and gums of a mouth.

16. The dental composition of claim 15, wherein the treatment is at least one of cleaning teeth, whitening teeth, improving gum disorders, removing dental biofilm from the mouth, and removing tartar from the mouth.

17. A dental composition for removing and inhibiting at least one of dental biofilm, plaque and tartar from a mouth, comprising an effective amount of ascorbic acid.

18. The dental composition of claim 17, wherein the composition further treats teeth and gum disorders, prevents teeth and gum disorders or both.

19. A dental method comprising the steps of:
(a) taking into a mouth an effective amount of a dental composition comprising ascorbic acid;
(b) maintaining the dental composition in the mouth for a sufficient period of time; and
(c) expectorating the dental composition from the mouth.

20. The dental method of claim 19, further comprising the steps of:
(a) rinsing the mouth with a rinse; and
(b) expectorating the rinse from the mouth.

21. The dental method of claim 19, further comprising repeating steps (a), (b) and (c) to inhibit the formation of dental biofilm and tartar in the mouth.

22. The dental method of claim 19, further comprising the step of using a dental tool to remove dental biofilm and tartar from the mouth.

23. The method as claimed in claim 22, wherein the dental tool is selected from the group consisting of a toothbrush, tooth strips, dental floss and dental instruments.

24. The method as claimed in claim 22, wherein the dental tool is impregnated with the composition.

25. A method of treating gum disorders, preventing gum disorders or both, comprising the steps of:
(a) taking into a mouth an effective amount of a dental composition comprising ascorbic acid;
(b) maintaining the dental composition in the mouth for a sufficient period of time; and
(c) expectorating the dental composition from the mouth.

26. The method of claim 25, further comprising the step of using a dental tool to remove dental biofilm, plaque and tartar from the mouth.
